(19) United States
(12) Patent Application Publication
(10) Pub. No.: US 2006/0105989 A1
(43) Pub. Date: May 18, 2006

(54) Food for improving arthritis

(76) Inventor: Kinya Takagaki, Fukuoka (JP)

Correspondence Address: AMIN & TUROCY, LLP, 1900 EAST 9TH STREET, NATIONAL CITY CENTER, 24TH FLOOR,, CLEVELAND, OH, 44114, US

(21) Appl. No.: 10/992,055

(22) Filed: Nov. 18, 2004

(51) Int. Cl. A61K 31/737; A61K 31/7008; A61K 31/353; A61K 31/7048

(52) U.S. Cl. 514/54; 514/62; 514/456

(57) ABSTRACT

The present invention provides a food for improving arthritis comprising proanthocyanidins (A) and a mucopolysaccharide and/or an amino sugar (B). Preferably, the proanthocyanidins contain at least 20 wt % of OPCs.

### BACKGROUND OF THE INVENTION

1. Field of the Invention

The present invention relates to a food for improving arthritis that comprises proanthocyanidins and a mucopolysaccharide and/or an amino sugar.

2. Description of the Related Art

With the aging of the population, the number of the people who suffer from arthritis such as knee pain and elbow pain has been increasing, and pharmaceutical products and health food products for alleviating such arthritis have been commercially available. Examples of these products include foods containing a mucopolysaccharide or an amino sugar, such as chondroitin and glucosamine, alone. Furthermore, the examples include foods containing a combination of glucosamine and chondroitin as described in Japanese Patent No. 2971579. The effect of these foods is widely recognized, but the effect is achieved slowly and cannot be experienced in a month or so after the start of ingestion of the foods. Thus, people often give up ingesting the above-described foods before they experience the effect of these foods.

Therefore, there is a demand for improving slow-acting properties of the conventional foods that have been used against arthralgia and the like. In other words, there is a demand for providing a food by which the effect of alleviating arthralgia can be experienced in a short period of time.

### SUMMARY OF THE INVENTION

The present invention provides a food for improving arthritis that comprises a proanthocyanidin (A) and a mucopolysaccharide and/or an amino sugar (B). By ingestion of this food, an effect of alleviating arthralgia can be experienced in a short period of time.

In a preferred embodiment, the amino sugar is glucosamine or a derivative thereof, or their salt.

In another preferred embodiment, the proanthocyanidin (A) comprises at least 20 wt % of OPC (oligomeric proanthocyanidin).

According to the present invention, it was found that by ingestion of the food that contains proanthocyanidins (A) and a mucopolysaccharide and/or an amino sugar (B), arthralgia could be alleviated immediately. In particular, when employing proanthocyanidins that contain at least 20 wt % of OPCs, arthralgia can be alleviated effectively.

### DESCRIPTION OF THE PREFERRED EMBODIMENT

Hereinafter, the food for improving arthritis of the present invention will be described. It should be noted that the following description is not limiting the present invention, and it is apparent to those skilled in the art that various alternations can be made within the scope of the spirit of the present invention.

The food for improving arthritis of the present invention comprises proanthocyanidins (A) and a mucopolysaccharide and/or an amino sugar (B). Hereinafter, these components of the food will be described.

(A) Proanthocyanidins

In the present invention, proanthocyanidins refer to a group of compounds that are condensation products having flavan-3-ol and/or flavan-3,4-diol as a constituent unit and having a degree of polymerization of 2 or more. Proanthocyanidins are known to have various activities such as an antioxidation ability.

For the proanthocyanidins contained in the food of the present invention, foodstuff raw materials such as ground products or extracts obtained from the fruit or seeds of grape, blueberry, strawberry, avocado, and the like can be used. In particular, it is preferable to use a pine bark extract. Among proanthocyanidins, OPCs are especially abundant in pine bark, and thus, a pine bark extract is preferably used as a raw material of the proanthocyanidins in the present invention.

Hereinafter, a method for preparing proanthocyanidins will be described taking a pine bark extract that contains OPCs abundantly as an example.

As the pine bark extract, an extract from the bark of plant belonging to Pinales, such as French maritime pine (Pinus martima), Larix leptolepis, Pinus thunbergii, Pinus densiflora, Pinus parviflora, Pinus pentaphylla, Pinus koraiensis, Pinus pumila, Pinus luchuensis, utsukushimatsu (Pinus densiflora form. umbraculifera), Pinus palustris, Pinus bungeana, and Anneda in Quebec, Canada, can be preferably used. Among these, French maritime pine (Pinus martima) bark extract is preferable.

French maritime pine refers to maritime pines that grow in a part of the Atlantic coastal area in southern France. It is known that the bark of this French maritime pine contains proanthocyanidins, organic acids, and other bioactive substances, and proanthocyanidins from the flavonoid family, which are the main component of the French maritime pine bark, have a potent antioxidation ability of removing active oxygen.

The pine bark extract is obtained by extracting the bark of the above-described pines using water or an organic solvent. When water is used, warm water or hot water can be employed. As the organic solvent that can be employed for extraction, an organic solvent that is acceptable for production of foods or pharmaceuticals can be employed. Examples of such solvent include methanol, ethanol, 1-propanol, 2-propanol, 1-butanol, 2-butanol, butane, acetone, hexane, cyclohexane, propylene glycol, aqueous ethanol, aqueous propylene glycol, methyl ethyl ketone, glycerin, methyl acetate, ethyl acetate, diethyl ether, dichloromethane, edible oils or fats, 1,1,1,2-tetrafluoroethane, and 1,1,2-trichloroethene. The water and the organic solvents may be used alone or in combination. In particular, hot water, aqueous ethanol, and aqueous propylene glycol are preferably used.

The method for extracting proanthocyanidins from pine bark is not particularly limited, and heat extraction or supercritical fluid extraction can be employed, for example.

Supercritical fluid extraction is a method for performing extraction using a supercritical fluid. A supercritical fluid is in a state that is above the liquid-vapor critical point in the phase diagram showing critical temperature and critical pressure. Examples of compounds that can be employed as a supercritical fluid include carbon dioxide, ethylene, propane, and nitrous oxide (laughter gas). Carbon dioxide is preferably used.

Supercritical fluid extraction includes an extraction step in which a target component is extracted with a supercritical fluid and a separation step in which the target component is separated from the supercritical fluid. In the separation step, any separation process can be employed, examples of which include a separation based on a change in pressure, a separation based on a change in temperature, and a separation using an adsorbent or absorbent.

Moreover, it is also possible to perform supercritical fluid extraction in which an entrainer is added. In this method, extraction is performed using an extracting fluid obtained by adding, for example, ethanol, propanol, n-hexane, acetone, toluene, or another aliphatic lower alcohol, aliphatic hydrocarbon, aromatic hydrocarbon, or ketone at about 2 to 20 W/V % to a supercritical fluid, so that the solubility of a target substance to be extracted, such as OPCs and catechins (described later), in the extracting fluid is dramatically increased or the selectivity of separation is enhanced. Thus, a pine bark extract is obtained efficiently.

Since supercritical fluid extraction can be performed at a relatively low temperature, it has the following advantages: it is applicable for extracting substances that deteriorate or decompose at high temperatures; the extracting fluid does not remain; and the extracting fluid can be recovered and recycled, so that a step of removing the extracting fluid and the like can be omitted, and thus, the process can be simplified.

Furthermore, methods other than those mentioned above can be employed for extraction from pine bark, and examples thereof include a batch method using liquid carbon dioxide, a reflux method using liquid carbon dioxide, a reflux method using supercritical carbon dioxide and the like.

It is also possible to employ a combination of a plurality of extraction processes to perform extraction from pine bark. By combining a plurality of extraction processes, pine bark extracts with various components can be obtained.

The pine bark extract that is used for the food of the present invention is specifically prepared using the following method. However, this method is merely an example, and the pine bark extract used for the present invention is not limited to the extract obtained by this method.

First, 1 kg of the bark of French maritime pine is immersed in 3 L of a saturated solution of sodium chloride, and extraction is performed for 30 minutes at 100° C. to obtain an extract liquid (extraction step). Then, the extract liquid is filtrated, and the resultant insoluble material is washed with 500 ml of a saturated solution of sodium chloride to obtain a washed liquid (washing step). The extract liquid and the washed liquid are combined to obtain a crude extract liquid of pine bark.

Next, 250 ml of ethyl acetate is added to this crude extract liquid, mixed, and separated to obtain an ethyl acetate layer. This process is repeated five times, and the obtained ethyl acetate layers are combined. The resultant ethyl acetate extract is added directly to 200 g of anhydrous sodium sulfate for drying. Then, this ethyl acetate extract is filtrated, and the filtrated extract is concentrated under a reduced pressure to a volume of 1/5 of the original filtrated extract. The concentrated ethyl acetate extract is poured into 2 L of chloroform and stirred, and the resultant precipitate is recovered by filtration. Subsequently, this precipitate is dissolved in 100 ml of ethyl acetate, and then the resultant solution is added to 1 L of chloroform to form a precipitate. This process is repeated twice, and thus, a washing process is accomplished. With this method, for example, about 5 g of pine bark extract containing at least 20 wt % of OPCs that have a degree of polymerization of 2 to 4 and at least 5 wt % of catechins can be obtained.

Extracts from the above-described raw material plants, in particular, pine bark extracts, which are typically used for the proanthocyanidins (A) in the food of the present invention, contain proanthocyanidins that are condensation products having flavan-3-ol and/or flavan-3,4-diol as a constituent unit and having a degree of polymerization of 2 or more. Among these, extracts that contain a large amount of condensation products having a lower degree of polymerization are preferably used. As such condensation products, condensation products having a degree of polymerization of 2 to 30 (dimer to 30-mer) are preferable, condensation products having a degree of polymerization of 2 to 10 (dimer to decamer) are more preferable, and condensation products having a degree of polymerization of 2 to 4 (dimer to tetramer; i.e., OPCs) are even more preferable.

Since OPCs are antioxidants as described above, they also provide an effect of reducing the possibility of adult diseases, such as cancer, cardiac diseases, and cerebral thrombosis, an effect of improving allergic diathesis, such as arthritis, atopic dermatitis, and pollenosis, and the like.

Furthermore, it is known that in addition to the antioxidation effect, OPCs also provide, for example, an effect of inhibiting bacterial proliferation in the oral cavity to reduce plaque (dental plaque); an effect of recovering the elasticity of blood vessels; an effect of preventing lipoprotein in blood from being damaged by active oxygen, thereby preventing aggregation and adherence of the oxidized fats onto the inside wall of the vessel, thus preventing cholesterol from being aggregated and adhered onto the oxidized fats that have been adhered onto the inside wall of the vessel; an effect of regenerating vitamin E that has been degraded by active oxygen; and an effect of serving as an enhancer of vitamin E.

In the present invention, proanthocyanidins containing at least 20 wt % of OPCs are preferably used. More preferably, the OPC content is at least 30 wt %. For such proanthocyanidins, a pine bark extract is preferably used.

When proanthocyanidins having a high OPC content are used, a better effect of alleviating arthralgia can be achieved than in the case where proanthocyanidins having a higher degree of polymerization (having a lower OPC content) are used.

Moreover, proanthocyanidin contents derived from raw material plants, in particular, plant extracts contain catechins as well as OPCs. The term “catechins” is a general term referring to polyhydroxyflavan-3-ols. As the catechins, for example, (+)-catechin, (−)-epicatechin, (+)-gallocatechin, (−)-epigallocatechin, epigallocatechin gallate, and epicatechin gallate are known. Gallocatechin, afzelechin, and 3-galloyl derivatives of (+)-catechin or gallocatechin are isolated from natural products, in addition to (+)-catechin that is called catechin in a narrow sense. Catechins are known to have a cancer inhibiting ability, an arteriosclerosis preventing ability, a lipid metabolism disorder inhibiting ability, a blood pressure elevation inhibiting ability, a thrombosis preventing ability, an antiallergic ability, an antiviral ability, an antibacterial ability, a dental caries preventing ability, a halitosis preventing ability, an intestinal flora normalization ability, an active oxygen or free radical eliminating ability, an antioxidation ability, and the like. Moreover, catechins are known to have an antidiabetic ability that inhibits an elevation of blood glucose. Furthermore, catechins have the property of both increasing the solubility in water and being activated in the presence of OPCs.

It is preferable that catechins are contained in the above-described raw material plant extract in a ratio of 5 wt % or more. Alternatively, it is also preferable that a formulation is prepared so that it contains a raw material plant extract containing at least 20 wt % of OPCs and furthermore, contains catechins in a ratio of 5 wt % or more. For example, when the catechin content in a pine bark extract is less than 5 wt %, it is possible to add catechins so that the catechin content becomes at least 5 wt %. It is most preferable to use a pine bark extract containing at least 5 wt % of catechins and at least 20 wt % of OPCs.

(B) Mucopolysaccharides and/or Amino Sugars

The food for improving arthritis of the present invention contains a mucopolysaccharide and/or an amino sugar. Mucopolysaccharides are known to provide an effect of improving arthritis, an effect of beautifying skin, and an effect of improving visual acuity. On the other hand, amino sugars are known to have an ability of improving arthritis, but there are few reports on other abilities.

Examples of mucopolysaccharides that can be used for the food of the present invention include hyaluronic acid, chondroitin, dermatan, heparan, heparin, and keratan, and their salts (e.g., sulfate and hydrochloride). A salt of hyaluronic acid or a salt of chondroitin can be preferably used. Also, food materials that contain these compounds abundantly, such as shark or bovine cartilage, chicken skin, swallow's nest, natto (fermented soybeans), and okra (Abelmoschus esculentus) can be used as materials containing mucopolysaccharide.

Examples of amino sugars include glucosamine, acetylglucosamine, galactosamine, acetylgalactosamine, neuraminic acid, acetylneuraminic acid, and hexosamine, and their salts (e.g., phosphates of such compounds). Also, food materials that contain these compounds abundantly, such as an extract from shark or bovine cartilage, can be preferably used as materials containing amino sugar.

Moreover, it is preferable that both the mucopolysaccharide and the amino sugar are contained. For example, an extract from shark or bovine cartilage, which contains both of them abundantly, is preferable.

The food for improving arthritis of the present invention comprises proanthocyanidins (A) and a mucopolysaccharide and/or an amino sugar (B) at a weight ratio of preferably 1:1 to 1:50 and more preferably 1:2 to 1:30.

The food for improving arthritis of the present invention contains the above-described proanthocyanidins (A) and mucopolysaccharide and/or amino sugar (B) and if necessary, comprises a variety of types of additives that are commonly used for foods. Examples of such additives include excipients, extenders, binders, thickeners, emulsifiers, coloring agents, flavors, and food additives. For example, the food of the present invention may be produced in the form of, for example, tablets or pills by adding an excipient and the like to a mixture of pine bark extract that contains proanthocyanidins abundantly and chondroitin sulfate and molding the mixture. Alternatively, the food may be produced in the form of powder or in other forms without being shaped. It is preferable to further add glucosamine.

It is also possible to employ the forms of capsules such as hard capsules and soft capsules, powder, granule, tea bags, candy, liquid, and paste.

Furthermore, nutritions, such as royal jelly, vitamins, proteins, calcium substances such as eggshell calcium, chitosan, lecithin, chlorella powder, Angelica keiskei powder, and molokheiya powder, also can be added. It is also possible to add stevia powder, ground green tea powder, lemon powder, honey, maltitol, lactose, sugar solutions, seasonings, and the like so as to control taste.

Regarding the method for ingesting the food for improving arthritis of the present invention, there is no particular limitation. According to the form of the food of the present invention or according to the preference, the food may be eaten or drunk as it is, or may be dissolved in water, hot water, milk, or the like and drunk. Alternatively, a liquid containing the components of the food obtained by percolation may be drunk.

Although there is no limitation regarding the daily intake amount of the food for improving arthritis of the present invention, it is preferable that the daily intake amount of the proanthocyanidins (A) is 20 mg to 2000 mg, and the daily intake amount of the mucopolysaccharide as (B) is 50 mg to 2000 mg. The daily intake amount of the amino sugar as (B) is preferably 50 mg to 2000 mg. The food of the present invention can contain these components in any suitable ratio.

It seems that with the food of the present invention, the effect of alleviating arthralgia can be achieved in a relatively short period of time because the proanthocyanidins (A) can promote the effect of alleviating arthralgia that is provided by the mucopolysaccharide and/or the amino sugar (B).

### EXAMPLES

Hereinafter, the present invention will be described by way of examples. However, the present invention is not limited to these examples.

### Example 1

First, an ethanol extract of pine bark (trade name: Flavangenol, produced by TOYO SHINYAKU Co., Ltd.) containing 40 wt % of proanthocyanidins (OPC content: 20 wt % in the extract) and 5 wt % of catechins, glucosamine, and crystalline cellulose were mixed in a ratio shown in Table 1 below to obtain “Food 1”. Separately, maltitol, sucrose ester, silicon dioxide, and the like were mixed at an appropriate ratio, resulting in a molding agent that serves as an excipient and a lubricant. This molding agent was mixed with the Food 1 at an equal amount to produce tablets, each having a weight of 200 mg.

Then, five volunteers (aged 45 years or older) who felt pain in the knee and elbow joints ingested 12 tablets of the thus produced tablets daily for four weeks. After the start of the ingestion, the volunteers performed self-assessment of the parts (knee and elbow) of arthralgia every week by employing the following criteria: the arthralgia was “improved”, “slightly improved”, or “unchanged”.

Table 2 shows the results. The values in Table 2 indicate the number of volunteers who remarked that the arthralgia in at least one of the parts of the knee and the elbow was “improved” or “slightly improved” until the week in which the survey was conducted.

### Example 2

“Food 2” was produced in the same manner as mentioned above using chondroitin sulfate in place of glucosamine and using the components shown in Table 1. The “Food 2” also was formed into tablets, and the assessment of arthralgia was performed in the same manner as in Example 1. Table 2 also shows the results.

### Example 3

“Food 3” was produced in the same manner as mentioned above using the components shown in Table 1 that include both of glucosamine and chondroitin sulfate. The “Food 3” also was formed into tablets, and the assessment of arthralgia was performed in the same manner as in Example 1. Table 2 also shows the results.

### Comparative Examples 1 to 3

“Food 4” to “Food 6” containing only one of the components (A) and (B) were produced using the components shown in Table 1 below. These foods were formed into tablets, and the assessment of arthralgia was performed as in Example 1. Table 2 also shows the results.

TABLE 1

| | | Ex. 1 | Ex. 2 | Ex. 3 | Com. Ex. 1 | Com. Ex. 2 | Com. Ex. 3 |
|---|---|---|---|---|---|---|---|
| Components | | Food 1 | Food 2 | Food 3 | Food 4 | Food 5 | Food 6 |
| (A) | Pine bark extract | 20 | 20 | 20 | 20 | — | — |
| (B) | Glucosamine | 60 | — | 60 | — | 60 | — |
| | Chondroitin sulfate | — | 20 | 20 | — | — | 20 |
| Additive | Crystalline cellulose | 20 | 60 | — | 80 | 40 | 80 |

The unit of the values is parts by weight.

TABLE 2

| | | | | Com. | Com. | Com. |
|---|---|---|---|---|---|---|
| | Ex. 1 | Ex. 2 | Ex. 3 | Ex. 1 | Ex. 2 | Ex. 3 |
| | Food 1 | Food 2 | Food 3 | Food 4 | Food 5 | Food 6 |
| 1 week | 0 | 0 | 1 | 0 | 0 | 0 |
| 2 weeks | 1 | 0 | 3 | 0 | 1 | 0 |
| 3 weeks | 2 | 2 | 4 | 0 | 1 | 0 |
| 4 weeks | 3 | 3 | 4 | 0 | 2 | 1 |

The values indicate the number of volunteers who remarked “improved” or “slightly improved”.

The results in Table 2 shows that when proanthocyanidins are employed in combination with an amino sugar or a mucopolysaccharide, arthralgia can be improved rapidly (Examples 1 and 2). Moreover, the results of Example 3 also show that when proanthocyanidins are employed in combination with both of a mucopolysaccharide and an amino sugar, the improvement can be achieved even faster.

The invention may be embodied in other forms without departing from the spirit or essential characteristics thereof. The embodiments disclosed in this specification are to be considered in all respects as illustrative and not limiting. The scope of the invention is indicated by the appended claims rather than by the foregoing description, and all changes which come within the meaning and range of equivalency of the claims are intended to be embraced therein.

What is claimed is:

1. A food for improving arthritis, comprising a proanthocyanidin (A) and a mucopolysaccharide and/or an amino sugar (B).

2. The food for improving arthritis of claim 1, wherein the amino sugar is glucosamine, a derivative of glucosamine, a salt of glucosamine, or a salt of the derivative.

3. The food for improving arthritis of claim 1, wherein the proanthocyanidin comprises at least 20 wt % of OPC (oligomeric proanthocyanidin).
